(19) United States
(12) Patent Application Publication
(10) Pub. No.: US 2002/0146380 A1
(43) Pub. Date: Oct. 10, 2002

(54) Depilatory composition

(75) Inventors: Hiromi Nambu, Wakayama (JP); Yuki Hidaka, Wakayama (JP); Zenbei Meiwa, Wakayama (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Kao Corporation, Tokyo (JP)

(21) Appl. No.: 10/053,658

(22) Filed: Jan. 24, 2002

(30) Foreign Application Priority Data
Jan. 26, 2001 (JP) 2001-18784
Oct. 11, 2001 (JP) 2001-313906

(51) Int. Cl. A61K 7/06; A61K 7/11; A61K 9/24

(52) U.S. Cl. 424/70.16

(57) ABSTRACT

A gel depilatory composition obtainable by mixing (a) a keratin reducing compound, (b) a hydrophilic high molecular compound having an ionic group, (c) a crosslinking agent and (d) water and has a high effect on the removal of body hairs and is decreased in odor peculiar to chemicals.

### INDUSTRIAL FIELD OF APPLICATION

The present invention relates to a depilatory composition effective for depilation and a depilatory gel sheet.

### PRIOR ARTS

A method of depilating using a keratin reducing compound such as thioglycolic acid as a depilatory dominates in recent years. These depilatories are made into preparations in the form of, mainly, a cream, a jelly or a mousse prior to use. Generally, these agents are wiped using tissues or nonwoven fabric and thereafter washed away after applied when they are used. Therefore, these depilatories have many problems that uniform application is difficult, hands are soiled by the chemicals when the these depilatories are applied, waiting time causes anxiety because no other works can be done during use, aftertreatments such as washing are troublesome and the odors (primarily a thio odor and a hydrogen sulfide odor) of the chemicals are strong.

In the publication of JP-A No. 61-221112, a “depilatory composition comprising a water-soluble high molecular compound and a depilatory as essential components” is disclosed, the composition being solidified into a film by removing water after it is applied to portions to be depilated and then being peeled off and removed. However, this composition when it is applied has the form of a liquid, paste or cream and the problems that uniform application is difficult, hands are soiled by the chemicals when the these depilatories are applied, waiting time causes anxiety because no other works can be done during use, and the odors (primarily a thio odor and a hydrogen sulfide odor) of the chemicals are strong are not therefore bettered. Further, this composition has additional problems that the peeling of the composition is accompanied by such a pain as to peel off an adhesive tape when it is peeled off, the time when depilation is finished is not unclear because the penetrative condition of the chemicals is not confirmed and there is a fear that film fragments are left on the skin.

Each publication of JP-A No. 62-10310 and JP-A No. 11-12123 discloses a “water activation depilatory sheet comprising a hydrophobic support having moisture-permeability and an applied layer containing a coating film-forming polymer and a depilating chemical”, the sheet being wetted with water prior to use, applied to portions to be depilated and peeled after dried to depilate. However, though these sheets improve the problems concerning applicability, waiting time and odors, the problems that the peeling of the composition is accompanied by such a pain as to peel off an adhesive tape when it is peeled off, the time when depilation is finished is not unclear because the penetrative condition of the chemicals is not confirmed and there is a fear that film fragments are left on the skin are not improved. Further, wetting the sheet with water is required in advance to use.

### SUMMARY OF INVENTION

It is an object of the present invention to provide a gel depilatory composition which can be treated in a short time by a simple operation of only applying a gel directly to the portions to be depilated, is free from anxiety concerning the odors of chemicals and can be thoroughly peeled off.

The invention provides a gel depilatory composition obtainable by mixing (a) a keratin reducing compound, (b) a hydrophilic high molecular compound having an ionic group, (c) a crosslinking agent and (d) water with one another.

The composition of the invention is preferred to have the component (b) crosslinked and be in the form of gel.

The invention provides a process for preparing the above shown composition by mixing or compounding (a), (b), (c) and (d) with one another.

It is preferable to mix (a) , (b) and (d) with one another and then to add (c) to the mixture to crosslink (b) and thereby gel the composition. Alternatively, after (c) is mixed with (b) and (d), (a) may be added by the time when the crosslinking of (b) is finished.

The hydrophilic high molecular compound (b) having an ionic group may be a water-absorptive high molecular compound or a water-soluble high molecular compound.

The composition may further contain (e) a nonionic hydrophilic high molecular compound, (f) water-insoluble particles and/or (g) a compound having the solubility parameter δ represented by the formula (I)in the range of 8 to 15:

$$δ = {( Δ E / V )}^{1 / 2} = {( {\sum }_{i} {Δ}_{{e}_{i}} / {\sum }_{i} {Δ}_{{V}_{i}} )}^{1 / 2} \qquad ( I )$$

where;

^{Δ}E: Cohesive energy density (cal/mol)

V: Molar volume (10⁻⁶ m³/mol)

Δeᵢ: Evaporation energy of an atom or an atomic group

^{Δ}vᵢ: Molar volume of an atom or an atomic group.

The invention moreover provides a depilatory gel sheet comprising a support and the gel depilatory composition as shown above, provided on and/or in the support.

<Detailed Description of Components>

(a) Keratin Reducing Compound

In the present invention, as a keratin reducing compound, at least one type selected from various compounds which reductively cut a keratin protein of body hairs may be used. Examples of such a keratin reducing compound may include thioglycolic acid, cysteine, their derivatives and salts of these compounds. Here, as the derivatives of thioglycolic acid, monoglyceryl thioglycolate may be exemplified and as the derivatives of cysteine, N-acylcysteine, particularly, N-acylcysteine in which the acyl group comprises an alkyloyl group having 1 to 10 carbon atoms may be exemplified. Also, as the salts of thioglycolic acid, cysteine or derivatives of them, ammonium salts, quaternary ammonium salts, alkali metal salts such as sodium salts and potassium salts, alkali earth metal salts such as calcium salts and magnesium salts, mono-, di-, tri-ethanol amine salts and basic amino acids of these compounds may be exemplified.

Further, as the keratin reducing compound, thioglyceryl alkyl ether represented by the formula (II):

HS—CH₂CH(OH)CH₂O[(CH₂)ₚO]_{q}R¹ (II)

wherein R¹ represents a straight-chain or branched alkyl group or an alkenyl group having 1 to 20 carbon atoms, p denotes an integer of 1 or more and preferably 1 to 3 and q is an integer of 0 or more and preferably 0 to 5, its derivatives and salts of these compounds may be used.

In thioglyceryl alkyl ether represented by the formula (II), as examples of preferable alkyl group R¹, straight-chain or branched alkyl groups having 1 to 20 carbon atoms, preferably 1 to 12 carbon atoms and more preferably 1 to 8 carbon atoms with the view of bettering penetration into hairs. Specific examples of thioglyceryl alkyl ether represented by the formula (II) may include ethoxyhydroxypropanethiol. Further, as its derivative, methoxyethoxyhydroxypropanethiol, ethoxyethoxyhydroxypropanethiol, propoxyethoxyhydroxypropanethiol and isopropoxyethoxyhydroxypropanethiol may be exemplified.

Moreover, as the keratin reducing compound, mercaptoalkylamides represented by the formula (III):

HS—(CH₂)ᵣNHCOR² (III)

wherein r denotes an integer from 1 to 5 and R² represents a straight-chain or branched alkyl group, alkenyl group, hydroxyalkyl group, aminoalkyl group or sugar residue having 1 to 19 carbon atoms, their derivatives and compounds selected from these salts may also be used.

In mercaptoalkylamides represented by the formula (III) as preferable examples of R², straight-chain or branched alkyl or alkenyl groups having 1 to 19 carbon atoms, preferably 1 to 11 carbon atoms and more preferably 1 to 7 carbon atoms with the view of bettering penetration into hairs. As mercaptoalkylamide represented by the formula (III), mercaptoethylgluconamide and 2-amino-N-(2-mercaptoethyl)propionamide may be exemplified.

Among these keratin reducing compounds, one or more types selected from thioglycolic acid, cysteine and their derivatives and salts of these compounds are preferable, one or more types selected from thioglycolic acid and its derivatives and salts of these compounds are more preferable and thioglycolic acid or its salts are particularly preferable.

The amount of the keratin reducing compound to be compounded in the gel depilatory composition of the present invention is preferably 0.5 to 30% by weight and more preferably 1 to 20% by weight in the gel depilatory composition with the view of obtaining a sufficient depilating ability and preventing a rough dry skin, though it is properly determined, for example, depending on the type of the keratin reducing compound and on whether alcohols, as are explained later, are present or not.

(b) Hydrophilic High Molecular Compound Having an Ionic Group

In the present invention, the “hydrophilic high molecular compound” means a water-absorptive high molecular compound and a water-soluble high molecular compound.

Here, the water-absorptive high molecular compound is a high molecular compound which can absorb and keep water in an amount one or more times the own weight in the following measuring method.

<Method of Measuring the Amount of Water to be Kept by the Water-absorptive High Molecular Compound>

1 g of the water-absorptive high molecular compound was swollen in 150 ml of physiological salt solution (0.9% NaCl solution, manufactured by Otsuka Pharmaceutical Co., Ltd.) for 30 minutes and then put into a nonwoven fabric bag, which was then dehydrated using a centrifuge under a load of 143 G for 10 minutes to measure the total weight (whole weight) after dehydrated. Then, the amount of water kept after the centrifugal dehydration was measured according to the following formula.

Amount of water kept after centrifugal dehydration (g/g)={(Whole weight)−(Weight of the nonwoven fabric bag)−(Weight of the water-absorptive high molecular compound)−(Amount of the liquid left in the nonwoven fabric bag)}/(Weight of the water-absorptive high molecular compound)

Also, the water-soluble high molecular compound means a high molecular compound having a solubility of 1% by weight in 25° C. water. This solubility can be confirmed by the fact that when an aqueous solution of 1% by weight of the water-soluble high molecular compound is placed in a quartz cell having an optical path of 10 mm to measure each transmittance of a light having a wavelength of 500 nm and a light having a wavelength of 800 nm, at least one of these transmittances is 60% or more.

In the present invention, the ionic group means an ionic functional group, specifically, a cationic or anionic functional group and preferably an anionic functional group. Examples of the anionic functional group include a carboxy group, sulfuric acid group, sulfonic group, phosphoric acid group, phosphoric group or salts of these groups. Among these groups, a carboxy group or its salt is preferable.

Examples of the hydrophilic high molecular compound having an ionic group in the present invention include derivatives of polyvinyl alcohol (e.g., itaconic acid-modified polyvinyl alcohol, sulfonic acid-modified polyvinyl alcohol and maleic acid-modified polyvinyl alcohol) or crosslinked compounds of these derivatives, poly(meth)acrylic acid and/or its salts or copolymers of these compounds [e.g., a (meth)acrylic acid/(meth)acrylate copolymer and/or its salt, (meth)acrylic acid/maleic acid copolymer and/or its salt and starch/(meth)acrylic acid graft copolymer or its salt] or crosslinked compounds of these compounds and semi-synthetic high molecular compound such as cellulose derivatives [carboxymethyl cellulose or its salt (e.g., a sodium salt and potassium salt)] or crosslinked compounds of these compounds, starch derivatives (e.g., carboxymethyl starch) and polysaccharide derivatives. One or more types among these compounds may be used.

Here, “(meth)acrylic acid” means both “acrylic acid” and “methacrylic acid”. Examples of the salt include alkali metal salts, ammonium salts, di- or mono-long chain alkyl or alkenyl type quaternary ammonium salts, tertiary amine salts, imidazoline salts, imidazolinium salts, amino acid type cationic surfactants, alkanolammoniums having 2 to 9 carbon atoms, alkyl or alkenyl substituted pyridiniums or basic amino acids. Among these compounds, ammonium salts, sodium salts, potassium salts and alkanolammonium salts having 2 to 9 total carbon atoms are preferable.

Among these compounds are preferable one or more types selected from derivatives of polyvinyl alcohol, such as itaconic acid-modified polyvinyl alcohol, sulfonic acid-modified polyvinyl alcohol and maleic acid-modified polyvinyl alcohol, poly(meth)acrylic acid and/or a salt thereof or a copolymer of these compounds, such as a (meth)acrylic acid/(meth)acrylic ester copolymer, a salt thereof, a (meth)acrylic acid/maleic acid copolymer, a salt thereof and a starch/acrylic acid graft copolymer and a salt thereof, and carboxymethyl cellulose and/or its salts are preferable, one or more types selected from poly(meth)acrylic acid and/or its salts, (meth)acrylic acid/(meth)acrylic ester copolymer and/or its salts, (meth)acrylic acid/maleic acid copolymer and/or its salts and carboxymethyl cellulose and/or its salts are more preferable and poly(meth)acrylic acid and/or its salts.

The molecular weight of the hydrophilic high molecular compound having an ionic group is preferably 10000 or more and more preferably 10000 to 10000000 in terms of average weight in view of easiness of gel formation and miscibility with depilatory chemicals. Also, as the hydrophilic high molecular compound having an ionic group, compounds having two types of molecular weight, for example, a molecular weight of 1000000 or less and a molecular weight exceeding 1000000 may be mixed. Mixing these two types makes it possible to provide the product with proper adhesiveness to the skin and is hence preferable.

The amount, to be compounded, of the hydrophilic high molecular compound having an ionic group is preferably 3 to 50% by weight, more preferably 5 to 30% by weight and particularly preferably 5 to 20% by weight in the gel depilatory composition from the view points of bettering the ability to retain water and other components such as depilatory chemicals, of improving the mechanical strength of the gel layer itself and of providing high flexibility to the gel layer.

(e) Nonionic Hydrophilic High Molecular Compound

The gel depilatory composition of the present invention is preferably compounded further of a nonionic hydrophilic high molecular compound to improve the stability of the gel.

The nonionic hydrophilic high molecular compound means a hydrophilic high molecular compound having no ionic group and the “hydrophilic high molecular compound” has the aforementioned meaning. Examples of such a nonionic hydrophilic high molecular compound include synthetic high molecular compounds such as polyvinyl alcohol, polyvinylpyrrolidone, polyacrylamide and polyethylene glycol, semi-synthetic high molecular compounds such as methyl cellulose, hydroxymethyl cellulose and hydroxyethyl cellulose and natural high molecular compounds such as polysaccharides (e.g., xanthane gum, oxypropylene-modified xanthane gum, tragacanth gum and guar gum), agar, gelatin, sea weeds and protein. The amount of such a nonionic hydrophilic high molecular compound to be compounded is preferably 0.1 to 10% by weight and more preferably 0.5 to 5% by weight in the composition. Also, the amount of the nonionic hydrophilic high molecular compound to be compounded is preferably 5 to 50 parts by weight and more preferably 5 to 20 parts by weight based on 100 parts by weight of the aforementioned hydrophilic high molecular compound having an ionic group.

(c) Crosslinking Agent

The gel depilatory composition of the present invention comprises a crosslinking agent to crosslink the hydrophilic high molecular compound having an ionic group and to improve the stability of the gel. The crosslinked gel depilatory composition is easily peeled off and is hence washed simply and the keratin reducing compound is scarcely left on the skin.

As the crosslinking agent, covalent-bonding crosslinking agents and ion-bonding crosslinking agents are exemplified and ion-bonding crosslinking agents are preferable. Examples of the covalent-bonding crosslinking agent include polyhydric alcohols such as di-alcohols and tri-alcohols; and difunctional materials such as dimethylolurea and epichlorohydrin. Examples of the ion-bonding crosslinking agents include polyvalent amines such as diamines, triamines and polyamines and salts of these amines; polyvalent metal hydroxides such as aluminum hydroxide, calcium hydroxide, magnesium hydroxide and zinc hydroxide; polyvalent metal oxides such as aluminum oxide, calcium oxide, magnesium oxide and zinc oxide; polyvalent metal inorganic salts such as aluminum sulfate, aluminum potassium sulfate, aluminum carbonate, aluminum chloride, calcium sulfate, calcium carbonate, calcium chloride, magnesium sulfate, magnesium carbonate, magnesium chloride, zinc sulfate, zinc carbonate, zinc chloride and aluminum methasilicate and polyvalent metal organic salts such as aluminum acetate, calcium acetate, magnesium acetate, zinc acetate, aluminum glycinate and an alkali earth metal salts of thioglycolic acid such as calcium or magnesium. The alkali earth metal salts of thioglycolic acid can double as the keratin reducing compound and the crosslinking agent.

Among the ion-bonding crosslinking agents, one or more types selected from polyvalent metal hydroxides, polyvalent metal oxides and polyvalent metal salts (polyvalent metal inorganic salts and polyvalent metal organic salts) are more preferable. Here, the polyvalence means two or more valences. Also, particularly, polyvalent metal hydroxides or polyvalent metal oxides are preferable because the gel can be made into a desired form and because of good workability during compounding since the time required for crosslinking, namely the time required for gelling is proper. Among the polyvalent metal hydroxides, aluminum hydroxides and calcium hydroxides are preferable because of easy availability and calcium hydroxide is most preferable.

The amount of the crosslinking agent to be compounded is preferably 0.05 to 10% by weight and more preferably 0.1 to 5% by weight in the gel depilatory composition from the viewpoints of crosslinking sufficiently, of bettering the ability to retain water and other components such as depilatory chemicals, of improving the mechanical strength of the gel layer itself and of providing the gel layer obtained by a proper closslinking with high flexibility though the amount varies depending on the type of the crosslinking agent.

The amount of the polyvalent metal of the polyvalent metal salt is preferably 0.001 to 2 equivalents, more preferably 0.01 to 1 equivalent and particularly preferably 0.05 to 1 equivalent to the ionic group of the hydrophilic high molecular compound having an ionic group. In this range, no liquid droplet is produced from the gel and the gel is not excessively hardened.

The crosslinking agent (c) may include also the following exmaples.

(f) Water-insoluble Particle

The gel depilatory composition of the present invention preferably comprises a water-insoluble particle. The term “water-insoluble” in the water-insoluble particle means that when one part by weight of the subject particle is dissolved in 99 parts by weight of water at 25° C., the solubility is 50% by weight or less. The solubility is calculated from the solid content when an aqueous solution is filtered through a filter paper (No. 2).

The water-insoluble particle to be used in the present invention is more preferably a water-insoluble organic particle and particularly preferably a water-insoluble organic polymer particle. It is to be noted that the water-insoluble particle according to the present invention excludes the aforementioned hydrophilic high molecular compounds having an ionic or nonionic group.

Examples of materials of the water-insoluble organic particle include particles of a water-insoluble organic polymer, wax, paraffin and fatty acid derivatives such as distearyl ether. Examples of materials of the water-insoluble organic polymer particle include synthetic polymers such as nylon, polyethylene, polypropylene, polyvinyl acetate, polyamide, polyethylene terephthalate, polystyrene, polyurethane and poly(meth)acrylate, copolymers or crosslinked materials of these compounds, rubbers such as ethylene rubber, propylene rubber, styrene/butadiene rubber, butadiene rubber and silicon rubber or crosslinked materials of these rubbers and natural polymers such as husks of fruits or their derivatives. Here, the “poly (meth)acrylic acid” involves both a “polyacrylic acid” and a “polymethacrylic acid”.

Among these materials, synthetic polymers such as polyethylene, polyamide, polystyrene, polyurethane and poly(meth)acrylate, copolymers or crosslinked materials of these polymers are preferable. These compounds may be used either singly or in combinations of two or more.

These water-insoluble organic particles may comprise inorganic materials to improve handling characteristics. Preferable examples of the inorganic material include bentonite, smectite, zeolite, talc, mica, kaolin, sepiolite, silica, calcium carbonate, aluminum oxide, titanium oxide, silicic acid anhydride and hydroxy ‘calcium’ apatite and other than the above compounds, nacreous materials. Among these materials, bentonite, talc, mica, kaolin and silica are preferably used.

The water-insoluble particle used in the present invention may be those obtained by granulating a primary particle into a secondary particle by using a binder. The binder may be either a water-soluble or water-insoluble binder.

Examples of materials of the water-soluble binder include synthetic products such as polyvinyl alcohol or its derivatives, poly(meth)acrylic acid and/or its salts, (meth)acrylic acid/(meth)acrylic ester copolymers and/or their salts, (meth)acrylic acid/maleic acid copolymers and/or their salts, polyvinylpyrrolidone and polyethylene glycol, semi-synthetic polymers such as methyl cellulose, salts of carboxymethyl cellulose (e.g., sodium salts and pottassium salts), hydroxyalkyl (e.g., methyl or ethyl) cellulose and starch derivatives (e.g., carboxymethyl starch) and natural polymers such as gelatin, agar, polysaccharides, sea weeds, vegetable adhesives and protein. Among these materials, polyvinylpyrrolidone and polyethylene glycol are preferable.

Examples of materials of the water-insoluble binder include fatty acid derivatives such as wax, paraffin and distearyl ether, synthetic polymers such as nylon, polyethylene, polypropylene, polyvinyl acetate, polyamide, polyethylene terephthalate, polystyrene, polyurethane and poly(meth)acrylate, copolymers or crosslinked materials of these polymers, poly(meth)acrylic acid and/or crosslinked materials of their salts or crosslinked materials of these copolymers [e.g., (meth)acrylic acid/(meth)acrylate copolymers and/or closslinked materials of their salts and (meth)acrylic acid/maleic acid copolymers and/or crosslinked materials of their salts of these copolymers], rubbers such as ethylene rubber, propylene rubber, styrene/butadiene rubber, butadiene rubber and silicon rubber or crosslinked materials of these rubbers and natural polymers such as chitosan, cellulose, ethyl cellulose, starch and husks of fruits or their derivatives.

Here, the salts mean alkali metal salts, alkali earth metal salts, ammonium salts and basic amino acid salts.

The water-insoluble particle and binder component to be used in the present invention may be the same materials or materials differing from each other.

The water-insoluble particle to be used in the present invention may be a globular, short-fiber-like or block particle. A porous particle may be used. Among these particles, globular or short-fiber-like particle is preferable and a globular particle is more preferable. In the case of a globular particle, the average particle diameter is preferably 5 to 500 μm, more preferably 5 to 300 μm and particularly preferably 50 to 300 μm. This average particle diameter is a volume average particle diameter measured by a scatter type particle distribution measuring meter LA-910 (Horiba, Ltd.). In the case of a short-fiber-like particle, the average length is preferably 150 μm to 2 mm and more preferably 200 μm to 1 mm. The average thickness is preferably 5 μm to 80 μm and more preferably 10 μm to 50 μm.

The hardness of the water-insoluble particle is preferably 10⁴ Pa or more in view of rolling easiness and moving easiness and more preferably 10⁵ to 10⁷ Pa because of low stimulation to the skin. The hardness was found by applying compressive pressure to one particle to measure a force required to deform 10% of the diameter. For measurement, a digital force gage (GX-0.2R, manufactured by NIDEC-SHIMPO corporation, minimum measuring load: 2 mN) and a test stand (FGS-50V-L, manufactured by NIDEC-SHIMPO corporation) were used to measure the force by applying compressive pressure to the particle at a rate of 10 mm/min at ambient temperature (20° C.) by using a plate probe.

In the present invention, needle-like or columnar crystals or water-insoluble inorganic particles selected from aggregates of these crystals may be used. Given as examples of such an inorganic particle are iron compounds, aluminum compounds, titanium compounds, silicon compounds, magnesium compounds and zinc compounds. Specific examples of the inorganic particle include wollastonite (CaSiO₃), potassium titanate (K₂Ti₆O₁₃), basic magnesium sulfate (MgSO₄.5 Mg(OH)₂.3H₂O), sepiolite (Mg₈Si₁₂O₃₀(OH)₄(OH₂)₄.8H₂O), zonotolite (6CaO.6SiO₂.H₂O), calcium carbonate (CaCO₃), zinc oxide (ZnO), magnesium hydroxide (Mg(OH)₂), aluminum borate (9Al₂O₃.2B₂O₂) and aluminosilicate [aM₂O/bAl₂O₃/cSiO₂/dRₘAₙ/yH₂O (wherein M represents Na and/or K, R represents one or more types selected from the group consisting of Na, K, Ca and Mg, A represents one or more types selected from the group consisting of CO₃, S₄, NO₃, OH and Cl, a is 1 to 6, b is 2 to 8, c is 2 to 12, d is 0 to 4, m is 1 to 2, n is 1 to 3 and y is 0 to 32)]. Among these compounds, aluminosilicate, wollastonite and zinc oxide are preferable in view of color tone, shape and proper hardness. These compounds may be used either singly or in combinations of two or more.

In the case where the inorganic particle is a crystal, its average length is preferably 0.1 to 50 μm and more preferably 1 to 20 μm. These needle-like or columnar crystals are preferably those forming an aggregate. As the shape of the aggregate, a tetra pot form as described in FIG. 7 of the publication of JP-A No. 11-228127, a whisker form (crystal whiskers) and a globular form (sea urchin from) (FIG. 6 in the same publication) are more preferable. The average particle diameter of such a crystal aggregate is preferably 0.5 to 300 μm and more preferably 1 to 250 μm. Here, the average length and the average particle diameter were calculated based on observation using a scanning type electron microscope.

These water-insoluble particle works to make the gel adhere to the fine irregularity of the skin thereby improving depilatory ability. The water-insoluble particle may be kneaded during the formation of the gel or dispersed on the gel stuck to nonwoven fabric and thereafter pressed to the gel thereby sticking the particle to the gel. The latter method in which the particle is dispersed on the gel and pressed is rather preferable because the effect of the particle is obtained more efficiently. Also, when the gel is wiped or rinsed off with water, the particle is rolled to thereby cut hairs from the roots of the hairs, showing a high depilatory effect.

The amount of the water-insoluble particle to be compounded is preferably 1 to 50% by weight, more preferably 3 to 30% by weight and particularly preferably 3 to 15% by weight in the gel depilatory composition of the present invention. (g) a compound having the solubility parameter δ represented by the formula (I) or a penetration promoter It is preferable to add a compound having the characteristics that the solubility parameter δ represented by the formula (I) is 8 to 15 as a penetration promoter to the gel depilatory composition of the present invention. The solubility parameter δ is a value given by Fedors.

$$δ = {( Δ E / V )}^{1 / 2} = {( {\sum }_{i} {Δ}_{{e}_{i}} / {\sum }_{i} {Δ}_{{V}_{i}} )}^{1 / 2} \qquad ( I )$$

where;

^{Δ}E: Cohesive energy density (cal/mol)

V: Molar volume (10⁻⁶ m³/mol)

^{Δ}eᵢ: Evaporation energy of an atom or an atomic group

^{Δ}Vᵢ: Molar volume of an atom or an atomic group.

As examples of such a compound, anyone of alcohols, ethers, ketones and esters may be given as far as it has a solubility parameter δ ranging from 8 to 15. Specific examples of the compound include benzyloxy ethanol (11.78), benzyl alcohol (12.53), propylene carbonate (11.18), dimethylformamide (12.12), methyl ethyl ketone (8.80), diethylene glycol monoethyl ether (10.78), 1-butanol (11.09), 1-propanol (11.54), 1-pentanol(10.76), 1,3-butanediol (14.46), 1,4-butanediol (14.65), diacetone alcohol (11.50), diethylene glycol diethyl ether (8.10), 1,8-pentanediol (12.45), diethylene glycol dibutyl ether (8.17), 1,2-hexanediol (13.15) and 1,3-dimethyl-2-imidazolidinone (12.10). Among these compounds, benzyloxy ethanol (11.78), benzyl alcohol (12.53), 1-butanol (11.09), 1-propanol (11.54), diethylene glycol diethyl ether (8.10), 1,2-hexanediol (13.15) and 1,8-pentanediol (12.45) are preferable in view of penetration-promoting effect and compounding stability (numerals in the parenthesis indicate a value of the solubility parameter δ).

These compounds having a solubility parameter δ of 8 to 15 may be used by combining one or more. A small amount of ethanol, propylene glycol or an emulsifier may be added to these compounds when these compounds are inferior in compatibility with the depilatory.

In the gel depilatory composition of the present invention, the amount of the compound having a solubility parameter δ of 8 to 15 is preferably 0.1 to 50% by weight, more preferably 1 to 20% by weight and particularly preferably 1 to 10% by weight.

Other Additives

In the depilatory composition of the present invention, a compound represented by the formula (IV) is preferably compounded irrespective of the value of the solubility parameter δ of the compound for the purpose of increasing the rate of penetration of the keratin reducing compound into body hairs to thereby improve depilating ability.

R³(OCₜH₂ₜ)ₛOH (IV)

wherein R³ represents an alkyl group having 3 or more carbon atoms, a phenyl group, a benzyl group, an alkoxyphenyl group, a naphthyl group or an alkoxynaphthyl group, s denotes 0 or 1 and t denotes an integer from 0 to 3.

In the compound represented by the formula (IV), as the alkyl group of R³, straight-chain or branched alkyl groups having preferably 3 to 8 carbon atoms and more preferably 3 to 6 carbon atoms may be exemplified in view of bettering penetration into hairs. Also as the alkoxy group constituting the alkoxyphenyl group or alkoxynaphthyl group of R³, the number of carbons is preferably 8 or less in the same manner as in the case of the alkyl group.

The amount of the compound represented by the formula (IV) in the depilatory composition of the present invention is preferably 0.1 to 50% by weight, more preferably 1 to 20% by weight and particularly preferably 1 to 10% by weight.

Further, at least one compound selected from straight-chain or branched monohydric alcohol, diol, triol and N-alkylpyrrolidone having 1 to 6 carbon atoms may be compounded for the same purpose. Among these compounds, methanol, ethanol and isopropanol may be exemplified as the monohydric alcohol, propylene glycol, 1,3-butylene glycol, isoprene glycol and hexylene glycol may be exemplified as the diol, glycerol may be exemplified as the triol and N-methylpyrrolidone may be exemplified as the N-alkylpyrrolidone. Among these compounds, ethanol, propylene glycol, 1,3-butylene glycol or N-methyl pyrrolidone is preferable.

Also, as an agent used to promote a depilating effect, ureas such as urea and thiourea or a compound having a guanidine residue such as guanidine hydrochloride and guanidine carbonate may be compounded in an amount of preferably 0.01 to 8% by weight and more preferably 0.1 to 5% by weight.

A plasticizer may be used to control the rate of the evaporation of water in the gel of the gel depilatory composition of the present invention and in view of flexibility and viscosity adjustment or various filler may be used with the view of reinforcing the gel.

Example of the plasticizer include polyhydric alcohols such as polyethylene glycol, propylene glycol, polypropylene glycol, glycerol, polyglycerol, sorbitol and 1,3-butanediol, ethers such as polyoxyethylene lauryl ether, polyoxyethylene cetyl ether and polyoxyethylene stearyl ether and esters such as sorbitan monolaurate, sorbitan trilaurate and polyoxyethylenesorbitan monolaurate. Among these compounds, propylene glycol and glycerol are preferable in view of simple preparation of the gel and stability. These plasticizers may be used either singly or in combinations of tow or more. The amount of the plasticizer to be added is preferably 1 to 50% by weight and more preferably 1 to 30% by weight in the total composition.

Examples of the filler include various organic or inorganic powders such as silica, alumina, talc, clay, kaolin, glass, ceramics, carbon and metal powders. The filler is preferably added in an amount of 0.01 to 5% by weight in the total composition.

<Support>

The support to be used as the depilatory gel sheet according to the present invention may have moisture-permeability, non-moisture-permeability or sparing moisture-permeability. The support may have any structure as far as it has water-resistance and can stand against application and peeling. Given as examples of the support having moisture-permeability are sheets or films, which may have some layers, such as woven fabrics, nonwoven fabrics, porous films and air-permeable films and laminates of these sheets or films. Because sheets are distinguished from films based only on the thickness of a layer, the films are assumed to be included in the sheets. A sheet made of nonwoven fabric is more preferable in view of moldability. The substrate is preferably a non-moisture-permeable substrate or a sparingly moisture-permeable substrate to restrain the odors of chemicals, to prevent dryness and to prevent the evaporative dissipation of effective components when the depilatory composition is used. As the support having non- or sparing-moisture-permeability, laminated sheets prepared by laminating polyethylene or the like on nonwoven fabric or paper are exemplified.

Examples of materials of the support include sheets using one or two or more types of various synthetic fibers such as polyester, nylon, Saran, polyethylene, polypropylene, polyvinyl chloride, polyvinylidene chloride, ethylene/vinyl acetate copolymers, ethylene/ethylacrylate copolymers, polytetrafluoroethylene, ionomer, polyacrylonitrile type resins, polyurethane, cellulose, rayon and acetates, paper and cloth.

The thickness of the support according to the present invention is preferably 0.01 to 5 mm, more preferably 0.1 to 3 mm and particularly preferably 0.1 to 2 mm.

<Gel Depilatory Composition and a Process for Preparing IT>

The gel depilatory composition of the present invention is a gel composition prepared by compounding (a) a keratin reducing compound, (b) a hydrophilic high molecular compound having an ionic group, (c) a crosslinking agent and (d) water, wherein the hydrophilic high molecular compound having an ionic group is crosslinked by the compounded crosslinking agent. The gel is one which takes a three-dimensional network or honeycomb structure in which sol dispersion particles are joined to each other by crosslinking or the like and has a solid form. The composition is in a gel state preferably at 5 to 50° C. and more preferably at 10 to 30° C.

The gel depilatory composition of the present invention -is obtainable by mixing the hydrophilic high molecular compound having an ionic group and the crosslinking agent to crosslink the hydrophilic high molecular compound during compounding and contains the keratin reducing compound and water. It is preferable to mix the keratin reducing compound, water and other components with the hydrophilic high molecular compound having an ionic group and then to add the crosslinking agent to crosslink the hydrophilic high molecular compound having an ionic group thereby gelling the composition. However, after the crosslinking agent is mixed with the hydrophilic high molecular compound having an ionic group and water, the keratin reducing compound and other components may be added by the time when the crosslinking of the hydrophilic high molecular compound having an ionic group is finished. Particularly, in the case of using a crosslinking agent which needs a considerable time for crosslinking, the latter method may be used.

The gel depilatory composition may be made into an optional form by the time when the crosslinking is finished. The hydrophilic high molecular compound having an ionic group may be a crosslinked product. In this case, the crosslinked products are bound by the crosslinking agent to gel. When the crosslinking agent is an ion bonding crosslinking agent, crosslinking can be attained by mixing it.

The water content of the gel depilatory composition of the present invention is preferably 30% by weight or more, more preferably 30 to 90% by weight and particularly preferably 30 to 85% by weight with the view of well retaining water and other components including depilatory chemicals, of improving the mechanical strength of the gel layer itself and also of improving the flexibility of the gel layer.

The water content in the present invention is found using the total weight and dry weight of the gel depilatory composition, which weights are measured by the following method, according to the following formula.

<Method of Measuring the Total Weight and Dry Weight of the Gel Depilatory Composition>

(1) About 3 g of a sample gel is exactly weighed and put on a thoroughly dried flat bottom dish having an inside diameter of about 50 mm and a height of about 30 mm. This weight is defined as the total weight of the gel depilatory composition.

(2) The sample is dried under heating for about 3 hours in a drier set to 105±2° C.

(3) After dried, the sample is allowed to cool to ambient temperature in a desiccator filled with a silica gel drying agent for about 30 minutes.

(4) The weight of the sample after dried is measured.

(5) The above steps (2) to (4) are repeated until the weight is constant. Heating time in this case is designed to be about one hour.

This constant weight is defined as the dry weight of the gel depilatory composition. Here, the constant weight is a weight when a difference in weight between the first measurement and the second measurement is 1% or less of the weight measured first.

Water content (wt%)=[{(Total weight of the gel depilatory composition)−(Dry weight of the gel depilatory composition)}/(Total weight of the gel depilatory composition)]×100.

The pH value of the gel depilatory composition of the present invention is determined appropriately corresponding to the type and the amount of keratin reducing compound to be compounded and based on whether alcohols are added or not. In general, in the case where thioglycolic acid or its salt is compounded in the depilatory composition of the present invention, the pH is preferably 8 to 13, more preferably 9 to 13 and particularly preferably 10 to 13 from the viewpoint of improving depilating ability and of decreasing odors of chemicals. The pH may be adjusted using, for example, potassium hydroxide or sodium hydroxide.

pH is measured by contacting a skin-check pH meter (Hanna Instruments) with a gel depilatory composition. The viscosity of the gel depilatory composition of the present invention, namely the value measured according to the following method is preferably 100000 mpa·s or more, more preferably 300000 mPa·s or more and particularly preferably 500000 mPa·s or more. The upper limit of the viscosity is preferably 20000000 mPa·s or less, more preferably 15000000 Mpa·s or less and particularly preferably 10000000 Mpa·s or less. In a viscosity range from 100000to 20000000mPa·s, neither any liquid droplet is produced from the gel nor the solid is too solidified and good moldability is obtained so that the composition is easily made into a sheet.

<Method of Measuring the Viscosity of the Gel Depilatory Composition>

All components are compounded and the viscosity of the gel depilatory composition 30 minutes after compounded was measured at 25° C. by using a helical viscometer (manufactured by TOKI SANGYO. CO. LTD.) in the condition of spindle Nos. T-F, T-D and T-E, a rotating speed of 0.5 to 5 r/min and 3 minutes.

The gel depilatory composition of the present invention may comprise components which are usually used for cosmetics according to the need. Examples of these components include surfactants, foaming promoters, solvents, polyols, lower alcohols, oils, thickeners, moisture retentive agents, wetting agents, feeling improvers, flame retardants, ultraviolet absorbers, antioxidants, antiseptics, disinfectants, antibacterial agents, powders, perfumes, dyes, coloring pigments, pearling agents, warm feeling agents, chelating agents, medical components, spraying agents, pH regulators and deodorants, which may be compounded to the extent that the effect of the present invention is not impaired.

Also, the raw materials to be compounded in the gel depilatory composition of the present invention are preferably reduced in the content of metals such as iron to prevent the deterioration of the hue and odor and the denaturalization of the keratin reducing material and to better the preservation stability.

No particular limitation is imposed on the dosage form of the gel depilatory composition of the present invention. Since the gel depilatory composition of the present invention is a gel, it may be used as it is. It may be also used as a depilatory gel sheet provided with the gel depilatory composition on and/or in a support according to the need. When the gel depilatory composition is provided on the support, there is a method in which the gel depilatory composition is stuck to a sheet such as a nonwoven fabric sheet. Also, when the gel depilatory composition is provided in the support, there is a method in which a solution of the hydrophilic high molecular compound having an ionic group which compound includes the keratin reducing compound and the crosslinking agent is stuck to a sheet of, for example, nonwoven fabric prior to gelation and fixed to the sheet.

A structure in which a non-moisture-permeable peelable film is applied to the surface of the gel stuck or fused to the sheet of, for example, nonwoven fabric and the product is put into a closed bag, such as a pouch or a pillow, which are consisted of non-penetrative laminate or the like, and can be opened and closed is preferable because the stability of the gel can be kept. When used, the gel depilatory composition is taken out from the closed bag, the peelable film is peeled off and the gel depilatory composition can be applied directly to the portions to be depilated. The operation is therefore easiest, which is desirable.

Also, when a sheet is used, the gel depilatory composition is preferably arranged such that all or a part of the periphery of the sheet or four corners of the sheet are left uncoated. This is preferable in view of operations because no hand is soiled. When the gel depilatory composition is present on a sheet, the thickness of the gel depilatory composition is preferably 0.01 to 5 mm and more preferably 0.05 to 3 mm. Also, when the gel depilatory composition is used as it is, its thickness is preferably 0.5 to 10 mm and more preferably 1 to 5 mm.

[Method of Using the Gel Depilatory Composition]

When the gel depilatory composition of the present invention is used, the gel depilatory composition is applied to the portion to be depilated and allowed to stand for preferably 2 to 20 minutes and more preferably 3 to 15 minutes to allow the keratin reducing compound to penetrate into body hairs and to reduce the hairs. During the course of treatment, the gel may be peeled a little to confirm the swelling condition of the hairs. After that, the gel is peeled off and the agent is removed together with the hairs cut by washing and wiping.

The gel depilatory composition of the present invention can produce the effect of removing body hairs more greatly by physically warming the body hairs to be depilated by using, for example, a heater or a steaming towel or by allowing heating element, which is made to be contained in the gel depilatory composition, to generate heat thereby chemically warming the portion upon use before or after the sticking. The warming temperature is preferably 40 to 50° C.

As to the portions to be depilated, the gel depilatory composition may be applied to all of the portions, such as hands, legs, the sides and a bikini line for which commercially available depilatories are currently used. Further, the gel depilatory composition may be applied to a face (eyebrow, mustache and downy hair) because of simple operations and low odors.

The gel depilatory composition of the present invention is easily applied and cleaned, has a high effect on the removal of body hairs and is decreased in odor peculiar to chemicals. Also, no skilled operation is required since the gel is only applied to the portion to be depilated, so that anyone can depilate in the same way. Also, because the gel depilatory composition is highly adhesive to the skin, desired works can be carried out during treatment, so that waiting time causes no anxiety. In addition, the gel depilatory composition can be thoroughly peeled off without leaving any fragment of the chemicals and without any pain. Moreover, after the gel depilatory composition is peeled off once during the course of treatment to confirm the penetrative condition of the chemicals based on the swelling state of body hairs, it may be applied again.

### EXAMPLE

### Example 1

A keratin reducing compound (thioglycolic acid) shown in Table 1 was added to water and the pH of the solution was then adjusted to the values shown in Table 1 by using sodium hydroxide (this solution was designated as a solution A). Meanwhile, the above solution A was added to a mixture, obtained by adding aluminum hydroxide and a solvent (propylene glycol) to sodium polyacrylate, with stirring. The mixed solution was filled in a closed container and the gel was stabilized at 50° C. overnight (12 hours) in a thermostat to prepare a gel depilatory composition with a thickness of 2 mm.

The resulting gel depilatory composition with a thickness of 2 mm was cut up into a form of 2.5 cm×2.5 cm, which was then stuck to 0.5-mm-thick polypropylene nonwoven fabric of 3.0 cm×2.5 cm. The gel portion of this nonwoven fabric was applied to the portion to be depilated to evaluate depilating ability according to the following method. The results are shown in Table 1.

<Method of Evaluation of Depilatory Ability>

Hairs of the front shin portion of the leg of a male panelist (one panelist) were cut to an even length as long as 1 cm or less and the number of hairs having a length of 1 mm or more and 1 cm or less in a range of 2.5 cm×2.5 cm was counted. Next, the nonwoven fabric to which the gel depilatory composition was stuck was applied within this range and then peeled off after it was allowed to stand for 3 minutes, followed by water-scrubbing with cotton. After that, the number of hairs left undepilated and having a length of 1 mm or more was counted to calculate the rate of depilation according to the following formula. Also, each easiness of application and sticking, adhesiveness of the gel to the skin, odors and washing ability (rate of removal of the gel depilatory composition only by scrubbing) were evaluated.

Rate of depilation (%)=(Number of hairs before depilation−Number of hairs after depilation)×100/Number of hairs before depilation

### Example 2

Using each component shown in Table 1, a 5-mm-thick gel depilatory composition was prepared in the same manner as in Example 1. The resulting 5-mm-thick gel depilatory composition was cut up into a form of 2.5 cm×2.5 cm and the evaluation of depilating ability was made in the same manner as in Example 1 except that the gel depilatory composition was directly applied to the portion to be depilated. The results are shown Table 1.

### Examples 3 and 4

Using each component shown in Table 1, a solution of a depilatory composition was prepared in the same manner as in Example 1. Before the gelation of the gel depilatory composition was finished, the gel depilatory composition during the course of gelation was applied to 0.5-mm-thick polyethylene nonwoven fabric of 2.5 cm×2.5 cm and the gel was fixed to the nonwoven fabric at 50° C. overnight (12 hours) in a thermostat. The thickness of the gel was 0.3 mm and the gel was penetrated also into the nonwoven fabric and was present therein. This nonwoven fabric was applied to the portion to be depilated to evaluate the depilating ability in the same manner as in Example 1. The results are shown in Table 1.

### Example 5

Using components other than the water-insoluble particle (1) shown in Table 1, a 5-mm-thick gel depilatory composition was prepared in the same manner as in Example 1. The resulting 5-mm-thick gel depilatory composition was stuck to 0 5-mm-thick polyethylene nonwoven fabric of 2.5 cm×2.5 cm. Thereafter, the water-insoluble particle (1) was uniformly sprayed. The composition was compressed to a thickness of 4 mm and further allowed to stand overnight. This nonwoven fabric was to which this gel depilatory composition was stuck was applied to the portion to be depilated to evaluate the depilating ability in the same manner as in Example 1. The results are shown in Table 1.

It is to be noted that the water-insoluble particle (1) was produced in the following method.

<Method of Producing the Water-insoluble Particle (1)>

A beaker was charged with 500 ml of a 1% polyvinyl alcohol (Gosenol GH-17, manufactured by The Nippon Synthetic Chemical Industry Co., Ltd.) solution and the atmosphere in the beaker was sufficiently replaced with nitrogen. After that, a monomer solution consisting of 100 g of lauryl acrylate, 5 g of divinylbenzene and 1 g of lauroyl peroxide was added to the above component to run polymerization at 75° C. for 8 hours in a nitrogen atmosphere while stirring at 300 rpm. After the polymerization was finished, the reaction product was subjected to filtration, followed by washing with water and ethanol and by drying to obtain 95 g of perfectly spherical water-insoluble particle (1) (average particle diameter: 250 μm).

### Example 6

A polyvalent metal hydroxide (calcium hydroxide) shown in Table 1 was dispersed in a plasticizer (glycerol). Then, an aqueous solution of 5% water-soluble polymer (hydroxyethyl cellulose) was added and the mixture was stirred, to which were added a keratin reducing compound (thioglycolic acid) and water. Further, the resulting solution was adjusted to the pH shown in Table 1 by using sodium hydroxide. Finally, sodium polyacrylate was added to and mixed with the resulting solution to obtain a depilatory composition. This composition was applied to 0.5-mm-thick polypropylene nonwoven fabric of 3.0 cm×2.5 cm in a thickness of 1 mm. The product was put into a closed container, which was placed in a thermostat at 50° C. overnight (12 hours) to stabilize the gel. The gel portion of this nonwoven fabric was applied to the portion to be depilated to evaluate the depilating ability in the same manner as in Example 1. The results are shown in Table 1.

### Example 7

A depilatory composition having the percentage composition shown in Table 1 was prepared in the same manner as in Example 6 except that the polyvalent metal hydroxide was not used and calcium thioglycolate was used and the depilating ability of the composition was evaluated in the same manner. The results are shown in Table 1.

### Comparative Examples 1 to 3

Each component shown in Table 1 was mixed to prepare a depilatory composition solution. This depilatory composition solution was applied directly to the portion to be depilated without using nonwoven fabric to evaluate the depilating ability in the same manner as in Example 1. The results are shown in Table 1.

TABLE 1

| Example | | | | | | | Comparative Example | | |
|---|---|---|---|---|---|---|---|---|---|
| 1 | 2 | 3 | 4 | 5 | 6 | 7 | 1 | 2 | 3 |

Depilatory compo-
sition (wt %)

| Thioglycolic acid | 5.0 | 5.0 | 5.0 | 5.0 | 5.0 | 5.0 | — | 5.0 | 5.0 | 2.0 |
|---|---|---|---|---|---|---|---|---|---|---|
| Calcium thioglycolate | — | — | — | — | — | — | 3.0 | — | — | — |
| Propylene glycol | 5.0 | 5.0 | 3.0 | 3.0 | 3.0 | — | — | 5.0 | 5.0 | 5.0 |
| Glycerol | — | — | — | 2.0 | 2.0 | 30 | 10 | — | — | — |
| Sodium polyacrylate | 10 | 10 | 10 | 10 | 10 | 10 | 10 | — | 3.0 | 55 |
| (1) *¹ | | | | | | | | | | |
| Sodium polyacrylate | 10 | 5 | — | — | — | — | — | — | — | — |
| (2) *² | | | | | | | | | | |
| Water-insoluble | — | — | — | — | 15 | — | — | — | — | — |
| particle (1) | | | | | | | | | | |
| Sodium hydroxide | Proper | Proper | Proper | Proper | Proper | Proper | — | Proper | Proper | Proper |
| | amount | amount | amount | amount | amount | amount | — | amount | amount | amount |
| Guanidine carbonate | — | 5.0 | — | — | — | — | — | — | — | — |
| Benzyloxy ethanol | — | — | 2.0 | — | — | — | — | — | — | — |
| Hydroxyethyl cellu- | — | — | 1.0 | 1.0 | 1.0 | 2.0 | 2.0 | 3.0 | — | — |
| lose *³ | | | | | | | | | | |
| Aluminum hydroxide | 0.5 | 0.5 | 0.5 | 0.5 | 0.5 | — | — | — | — | — |
| Calcium hydroxide | — | — | — | — | — | 2.5 | — | — | — | — |
| Purified water | Balance | Balance | Balance | Balance | Balance | Balance | Balance | Balance | Balance | Balance |
| pH | 12.5 | 10.5 | 12.5 | 12.5 | 11.5 | 12.5 | 12.0 | 12.5 | 11.5 | 10.5 |
| Water content (m.Pa.s) | 60 | 66 | 69 | 73 | 56 | 51 | 75 | 82 | 82 | 36 |
| Viscosity 30 minutes | 10000000.0 | 5000000.0 | 1000000 | 8000000 | 7000000 | 3000000 | 1000000 | 10000 | 5000 | 30000000 |
| after compounded | | | | | | | | | | |
| (mPa · s) | | | | | | | | | | |
| Result of evaluation | | | | | | | | | | |
| Applying or sticking | Good | Good | Good | Good | Good | Good | Good | Difficult | Difficult | Difficult |
| easiness | | | | | | | | | | |
| Adhesiveness of the gel | Very good | Good | Good | Good | Good | Good | Good | Difficult | Difficult | Difficult |
| to the skin | | | | | | | | | | |
| Odor (during depilation) | acceptable | acceptable | acceptable | acceptable | acceptable | acceptable | acceptable | Unaccept- | Unaccept- | Unaccept- |
| | | | | | | | | able | able | able |
| | | | | | | | | strong | odor | strong |
| | | | | | | | | odor | | odor |
| Cleanability (remova- | Good | Good | Good | Good | Good | Good | Good | Inferior | Inferior | Inferior |
| bility of the depilatory | | | | | | | | | | |
| composition only by | | | | | | | | | | |
| scrubbing) | | | | | | | | | | |
| Rate of depilation (%) | 98 | 95 | 98 | 97 | 90 | 98 | 93 | 80 | 30 | 0 |

Note
*¹ Sodium polyacrylate (1): manufactured by Wako Pure Chemical Industries, Ltd., weight average molecular weight: 4400000.
*² Sodium polyacrylate (2): manufactured by Wako Pure Chemical Industries, Ltd., weight average molecular weight: 250000.
*³ Hydroxyethyl cellulose; manufactured by Daicel Chemical Industries, Ltd., HEC SP400.

1. A gel depilatory composition obtained by compounding (a) a keratin reducing compound, (b) a hydrophilic high molecular compound having an ionic group, (c) a crosslinking agent and (d) water.

2. The composition according to claim 1, wherein the crosslinking agent is an ionic bonding crosslinking agent.

3. The composition according to claim 1 or 2, which furhter comprises (e) a nonionic hydrophilic high molecular compound.

4. The composition according to claim 1 or 3, which further comprises (f) water-insoluble particles.

5. The composition according to claim 1, wherein (b) the hydrophilic high molecular compound having an ionic group is at least one selected from the group consisting of a poly(meth)acrylic acid and a salt thereof and (c) the crosslinking agent is at least one selected from the group consisting of a polyvalent metal salt, a polyvalent metal hydroxide and a polyvalent metal oxide.

6. The composition according to claim 1, having a viscosity of 100,000 Mpa·s or more.

7. The composition according to claim 1, in which (b) the hydrophilic high molecular compound having an ionic group is a water-absorptive high molecular compound or a water-soluble high molecular compound.

8. The composition according to claim 1, which further comprises (g) a compound having the solubility parameter δ represented by the formula (I)in the range of 8 to 15:
$$δ = {( Δ E / V )}^{1 / 2} = {( {\sum }_{i} {Δ}_{{e}_{i}} / {\sum }_{i} {Δ}_{{V}_{i}} )}^{1 / 2} \qquad ( I )$$
where;
^{Δ}E: Cohesive energy density (cal/mol)
V: Molar volume (10⁻⁶ m³/mol)
^{Δ}eᵢ: Evaporation energy of an atom or an atomic group
^{Δ}Vᵢ: Molar volume of an atom or an atomic group.

9. A depilatory gel sheet comprising a support and the gel depilatory composition as claimed in claim 1 to 6, provided on and/or in the support.
